(19) United States
(12) Patent Application Publication
(10) Pub. No.: US 2026/0240801 A1
(43) Pub. Date: Aug. 20, 2026

(54) COMPOSITIONS FOR TREATING PARKINSON’S DISEASE AND QUALITATIVE ASSESSMENT THEREOF

(71) Applicant: InTrance Medical Systems Inc., New York, NY (US)

(72) Inventors: Krai-Rith Chatamra, Libertyville, IL (US); Onkar N. Singh, Forth Worth, TX (US)

(21) Appl. No.: 19/161,948

(22) PCT Filed: Mar. 22, 2024
(86) PCT No.: PCT/US2024/021080

§ 371 Date: Sep. 3, 2025

Related U.S. Application Data
(60) Provisional application No. 63/492,365, filed on Mar. 27, 2023.

(51) Int. Cl. A61K 31/196; A61K 31/198; A61K 31/277; A61K 47/38

(52) U.S. Cl. CPC A61K 31/196; A61K 31/198; A61K 31/277; A61K 47/38

(57) ABSTRACT

The present disclosure provides compositions comprising Compound 1 uses thereof, and methods of manufacture thereof.

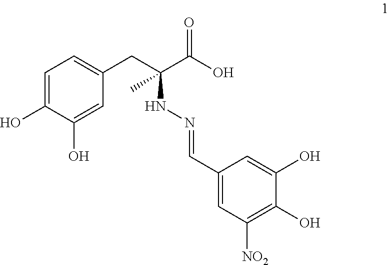 [SMILES: C[C@@](Cc1ccc(O)c(O)c1)(N/N=C/c1cc(O)c(O)c([N+](=O)[O-])c1)C(=O)O]

### CROSS REFERENCE TO RELATED APPLICATIONS

This application claims priority to U.S. Provisional Application No. 63/492,365 filed Mar. 27, 2023, the content of which is herein incorporated by reference in its entirety.

### BACKGROUND

Parkinson's disease is a neurological disorder causing involuntary movement in the body, including shaking, stiffness, and difficulty or loss of balance and coordination. As of 2016, approximately 6.1 million people have been affected by the disease. See Bloem, et al., The Lancet, 397(10291): 12-18 (2021). There is no cure for Parkinson's disease. A patient suffering from Parkinson's disease will face continuous progression of the disease throughout their life.

### SUMMARY

Particular treatment options are available to patients suffering from neurodegenerative disorders, such as Parkinson's disease, that help a patient manage symptoms, such as dyskinesia, involuntary movements, shaking, or stiffness. One such treatment is a combination of two or three of the following agents: carbidopa, entacapone, and levodopa. In some embodiments, carbidopa is carbidopa monohydrate.

Administration of these agents, however, has presented certain challenges, including particular stability challenges when administering carbidopa, entacapone, and levodopa in a single composition. Applicant discovered, however, that a particular compound, Compound 1, a by-product of carbidopa and entacapone, is formed during storage:

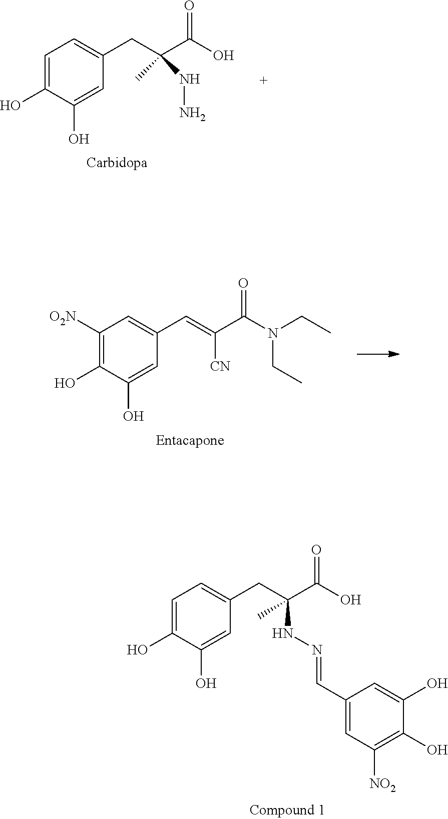 [SMILES: C[C@@](Cc1ccc(O)c(O)c1)(NN)C(=O)O ; CCN(CC)C(=O)/C(C#N)=C/c1cc(O)c(O)c([N+](=O)[O-])c1 ; C[C@@](Cc1ccc(O)c(O)c1)(N/N=C/c1cc(O)c(O)c([N+](=O)[O-])c1)C(=O)O]

Compound 1 is also known as (2s)-2-[(E)-2-[(3,4-dihydroxy-5-nitrophenyl)methylidene]hydrazin-1-yl]-3-(3,4-dihydroxyphenyl)-2-methylpropanoic acid

By discovering and characterizing Compound 1, Applicant has found, among other things, new compositions that can be used for treatment of Parkinson's disease, as well as discovered certain insights for qualitatively analyzing compositions comprising carbidopa and entacapone, which are useful for understanding and improving the stability of such compositions. The present application represents Applicant's identification of a source of a problem related to stability of certain compositions comprising carbidopa, entacapone, and levodopa, and presents herein certain solutions to that problem.

In some embodiments, the present disclosure provides a composition comprising greater than 0.15% by weight of Compound 1:

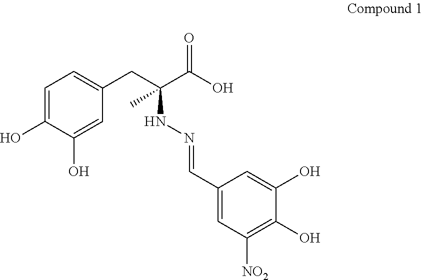 [SMILES: C[C@@](Cc1ccc(O)c(O)c1)(N/N=C/c1cc(O)c(O)c([N+](=O)[O-])c1)C(=O)O]

or a pharmaceutically acceptable salt thereof.

In some embodiments, the present disclosure provides a pharmaceutical composition comprising, carbidopa, entacapone, and greater than 0.15% by weight of Compound 1:

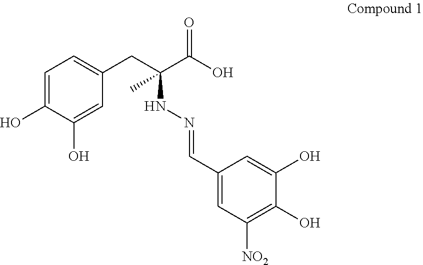 [SMILES: C[C@@](Cc1ccc(O)c(O)c1)(N/N=C/c1cc(O)c(O)c([N+](=O)[O-])c1)C(=O)O]

or a pharmaceutically acceptable salt thereof.

In some embodiments, the present disclosure provides a method of manufacturing a composition comprising Compound 1

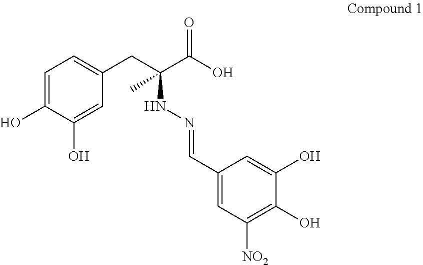 [SMILES: C[C@@](Cc1ccc(O)c(O)c1)(N/N=C/c1cc(O)c(O)c([N+](=O)[O-])c1)C(=O)O]

comprising contacting Compound 2 (i.e., 3,4-dihydroxy-5-nitrobenzaldehyde)

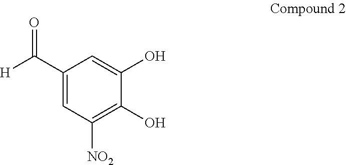 [SMILES: O=Cc1cc(O)c(O)c([N+](=O)[O-])c1]

with Compound 3 (i.e., S-carbidopa)

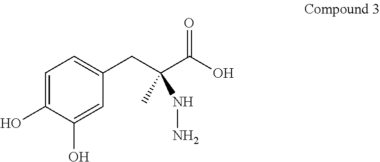 [SMILES: C[C@@](Cc1ccc(O)c(O)c1)(NN)C(=O)O]

in the presence of methanol and a sulfate salt.

In some embodiments, the present disclosure provides a method of characterizing the purity of a pharmaceutical composition comprising levodopa, carbidopa, and entacapone, the method comprising determining a quantity of Compound 1:

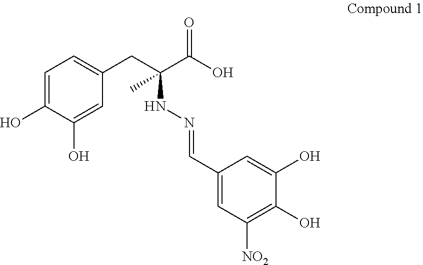 [SMILES: C[C@@](Cc1ccc(O)c(O)c1)(N/N=C/c1cc(O)c(O)c([N+](=O)[O-])c1)C(=O)O]

in the pharmaceutical composition by comparison of a sample of the pharmaceutical composition to a reference standard, wherein the reference standard comprises a known quantity of Compound 1.

In some embodiments, the present disclosure provides a method of using a reference standard comprising a known quantity of Compound 1:

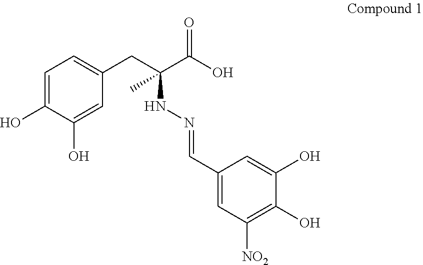 [SMILES: C[C@@](Cc1ccc(O)c(O)c1)(N/N=C/c1cc(O)c(O)c([N+](=O)[O-])c1)C(=O)O]

to determine a level of purity of a pharmaceutical composition, the method comprising comparing a quantity of Compound 1 in the reference standard to a quantity of Compound 1 in the pharmaceutical composition.

### BRIEF DESCRIPTION OF THE DRAWING

FIG. 1 is a plot measuring purity at 220 nm of composition comprising Compound 1 at indicated conditions for indicated time.

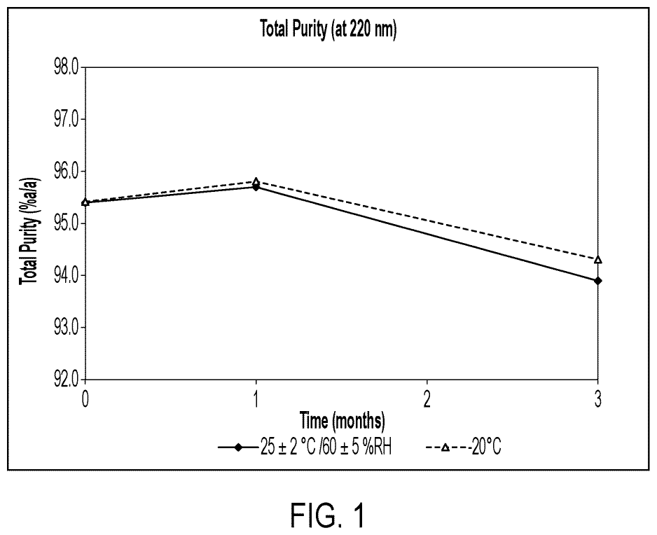

FIG. 2 is a plot measuring purity at 280 nm of composition comprising Compound 1 at indicated conditions for indicated time.

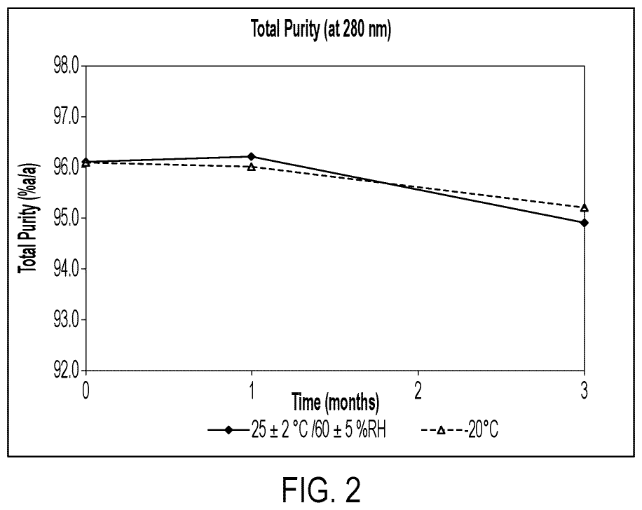

FIG. 3 is a plot measuring purity at 220 nm of composition comprising Compound 1 at indicated conditions for indicated time.

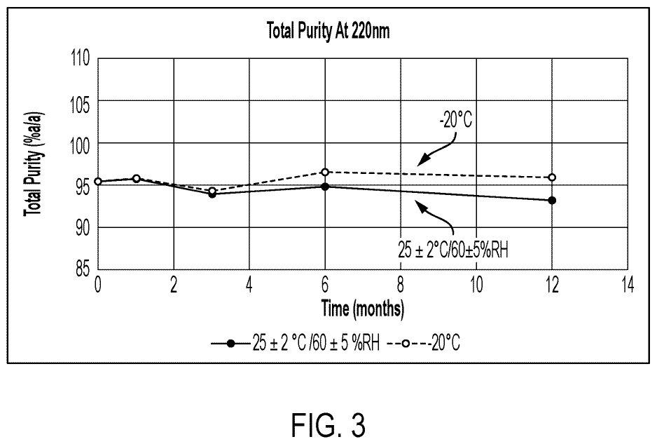

FIG. 4 is a plot measuring purity at 220 nm of composition comprising Compound 1 at indicated conditions for indicated time.

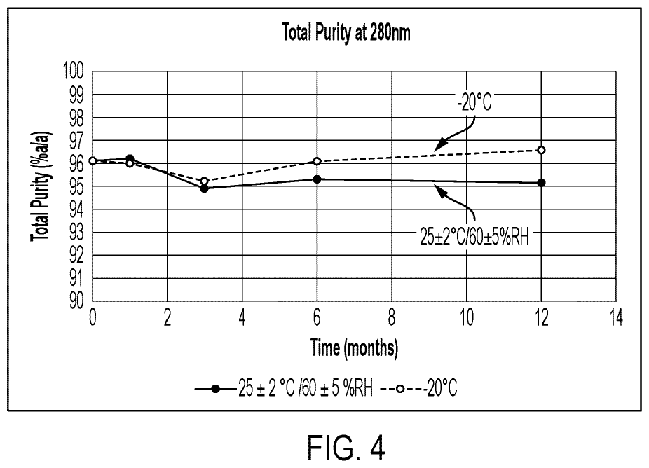

FIG. 5 is a plot measuring water content of composition comprising Compound 1 at indicated conditions for indicated time.

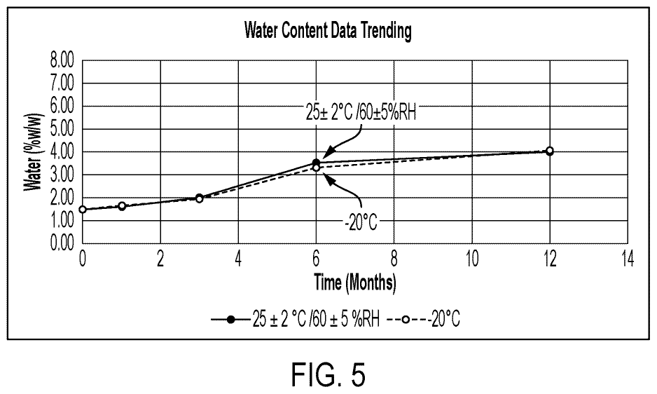

### DETAILED DESCRIPTION OF CERTAIN EMBODIMENTS

The present disclosure encompasses, among other things, an insight that certain by-products can form in compositions comprising carbidopa and entacapone, such as Compound 1:

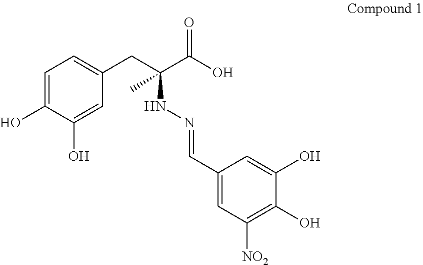 [SMILES: C[C@@](Cc1ccc(O)c(O)c1)(N/N=C/c1cc(O)c(O)c([N+](=O)[O-])c1)C(=O)O]

and such a discovery is implemented herein into, among other things, new compositions, new methods of qualifying compositions comprising carbidopa and entacapone, and new methods of treating patients suffering from neurodegenerative disorders, such as Parkinson's Disease.

### Definitions

About or approximately: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within (i.e., +) 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some particular embodiments, administration may be intravenous. In some particular embodiments, administration may be subcutaneous. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may comprise a prime-and-boost protocol. A prime-and-boost protocol can include administration of a first dose of a pharmaceutical composition (e.g., an immunogenic composition, e.g., a vaccine) followed by, after an interval of time, administration of a second or subsequent dose of a pharmaceutical composition (e.g., an immunogenic composition, e.g., a vaccine). In the case of an immunogenic composition, a prime-and-boost protocol can result in an increased immune response in a patient.

Antagonist: As will be understood by those skilled in the art, the term “antagonist” generally refers to an agent whose presence or level correlates with decreased level or activity of a target, as compared with that observed absent the agent (or with the agent at a different level). In some embodiments, an antagonist is one whose presence or level correlates with a target level or activity that is comparable to or less than a particular reference level or activity (e.g., that observed under appropriate reference conditions, such as presence of a known antagonist, e.g., a positive control). In some embodiments, an antagonist may be a direct antagonist in that it exerts its influence directly on (e.g., interacts directly with) the target; in some embodiments, an antagonist may be an indirect antagonist in that it exerts its influence indirectly (e.g., by acting on, such as interacting with, a regulator of the target, or with some other component or entity.

Biological sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination therapy: As used herein, the term “combination therapy” refers to those situations in which a subject is simultaneously exposed to two or more therapeutic regimens (e.g., two or more therapeutic agents or modality(ies)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, spray, gel, cream, ointment, solution, suspension, liquid, solid, etc.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, carboxy methyl cellulose (CMC), sodium carboxy methyl cellulose (NaCMC), hydroxyl ethyl cellulose (HEC), hydroxypropyl methyl cellulose (HPMC), carbomer, carbophil, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Modulator: The term “modulator,” as used herein, refers to a compound (e.g., a small molecule) that can alter the activity of another molecule (e.g., a protein). For example, in some embodiments, a modulator can cause an increase or decrease in the magnitude of a certain activity of a type of molecule as compared to the magnitude of the activity in the absence of the modulator. For example, a modulator can be an agonist or an antagonist of a particular target, as those terms are defined herein. For example, in some embodiments, a modulator is an agonist. In some embodiments, a modulator is an antagonist.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, intramuscular, subcuticular, intraarticulare, subcapsular, subarachnoid, intraspinal, and intrasternal injection and infusion.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amount appropriate for administration in a therapeutic or dosing regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), gel, tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977).

Prevent or prevention: As used herein, the terms “prevent” or “prevention”, when used in connection with the occurrence of a disease, disorder, and/or condition, refer to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid.

In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent.

Those of ordinary skill in the art, reading the present disclosure, will appreciate that certain small molecule compounds described herein may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more steroisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for ¹⁶O; ³⁶Cl for ³⁵Cl or ³⁷Cl; ¹⁸F for ¹⁹F; ¹³¹I for ¹²⁷I; etc.). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### Compound 1

In some embodiments, the present disclosure provides a composition comprising greater than 0.15% by weight of Compound 1

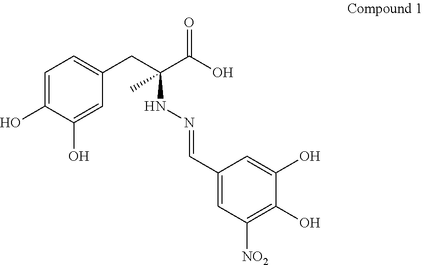 [SMILES: C[C@@](Cc1ccc(O)c(O)c1)(N/N=C/c1cc(O)c(O)c([N+](=O)[O-])c1)C(=O)O]

or a pharmaceutically acceptable salt thereof.

Applicant discovered that Compound 1 ((2s)-2-[(E)-2-[(3,4-Dihydroxy-5-nitrophenyl)ethylidene]hydrazin-1-yl]-3-(3,4-dihydroxyphenyl)-2-methylpropanoic acid) is a by-product of carbidopa and entacapone. Allowing a higher content of Compound 1 in a composition of carbidopa and entacapone prolongs the shelf-life of said composition. A composition comprising carbidopa and entacapone, in combination with levodopa, is a therapy useful for the treatment of Parkinson's Disease.

To date, two such products provide compositions having carbidopa, entacapone, and levodopa for treating patients suffering from Parkinson's Disease: Intrance Medical System's Lecigon®, and Novartis's Stalevo®. It has been observed, however, that administration of levodopa is challenging, due to the agent's poor bioavailability. It was discovered that by administering levodopa with entacapone, the efficacy of levodopa is increased substantially over therapies that lack entacapone, such as AbbVie's Duopa®, which only includes carbidopa and levodopa.

Current formulations comprising carbidopa and entacapone face relatively short shelf life, such as about 16 weeks at refrigerated conditions (e.g., 5±3° C.) due to the emergence of byproducts or other impurities. Current regulatory requirements do not allow for pharmaceutical compositions for treatment to have greater than 0.15% by weight of any impurity not identified in regulatory documents, however, and analyzed for safety. Applicant's identification and characterization of Compound 1 provides opportunities for new treatments that have longer shelf life, which ultimately will provide more access to therapies for patients.

Further, the discovery and characterization of Compound 1 provides manufacturers with a reference standard that can be used to qualify compositions comprising carbidopa and entacapone. Such compositions comprise Compound 1 and optionally, any other suitable solvent, diluent, carrier, or the like, to assist manufacturers in quality control.

In some embodiments, the present disclosure provides a composition comprising greater than 0.15% by weight of Compound 1:

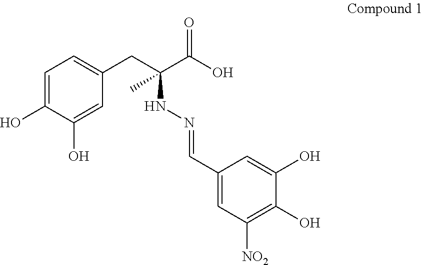 [SMILES: C[C@@](Cc1ccc(O)c(O)c1)(N/N=C/c1cc(O)c(O)c([N+](=O)[O-])c1)C(=O)O]

or a pharmaceutically acceptable salt thereof.

In some embodiments, a composition comprises greater than or equal to 1% by weight of Compound 1. In some embodiments, a composition comprises greater than or equal to 10% by weight of Compound 1. In some embodiments, a composition comprises greater than or equal to 15% by weight of Compound 1. In some embodiments, a composition comprises greater than or equal to 20% by weight of Compound 1. In some embodiments, a composition comprises greater than or equal to 25% by weight of Compound 1. In some embodiments, a composition comprises greater than or equal to 30% by weight of Compound 1. In some embodiments, a composition comprises greater than or equal to 35% by weight of Compound 1. In some embodiments, a composition comprises greater than or equal to 40% by weight of Compound 1. In some embodiments, a composition comprises greater than or equal to 45% by weight of Compound 1. In some embodiments, a composition comprises greater than or equal to 50% by weight of Compound 1. In some embodiments, a composition comprises greater than or equal to 55% by weight of Compound 1. In some embodiments, a composition comprises greater than or equal to 60% by weight of Compound 1. In some embodiments, a composition comprises greater than or equal to 65% by weight of Compound 1. In some embodiments, a composition comprises greater than or equal to 70% by weight of Compound 1. In some embodiments, a composition comprises greater than or equal to 75% by weight of Compound 1. In some embodiments, a composition comprises greater than or equal to 80% by weight of Compound 1. In some embodiments, a composition comprises greater than or equal to 85% by weight of Compound 1. In some embodiments, a composition comprises greater than or equal to 90% by weight of Compound 1. In some embodiments, a composition comprises greater than or equal to 95% by weight of Compound 1. In some embodiments, a composition comprises greater than or equal to 96% by weight of Compound 1. In some embodiments, a composition comprises greater than or equal to 97% by weight of Compound 1. In some embodiments, a composition comprises greater than or equal to 98% by weight of Compound 1. In some embodiments, a composition comprises greater than or equal to 99% by weight of Compound 1. In some embodiments, a composition consists essentially of Compound 1.

In some embodiments, a composition comprises less than 10% by weight of an organic solvent. In some embodiments, a composition comprises less than 9% by weight of an organic solvent. In some embodiments, a composition comprises less than 8% by weight of an organic solvent. In some embodiments, a composition comprises less than 7% by weight of an organic solvent. In some embodiments, a composition comprises less than 6% by weight of an organic solvent. In some embodiments, a composition comprises less than 5% by weight of an organic solvent. In some embodiments, a composition comprises less than 4% by weight of an organic solvent. In some embodiments, a composition comprises less than 3% by weight of an organic solvent. In some embodiments, a composition comprises less than 2% by weight of an organic solvent. In some embodiments, a composition comprises less than 1% by weight of an organic solvent.

In some embodiments, an organic solvent is selected from methanol, dichloromethane, acetone, ethanol, isopropanol, ethyl acetate, acetaldehyde, heptane, and acetaldehyde. In some embodiments, an organic solvent is methanol. In some embodiments, an organic solvent is dichloromethane. In some embodiments, an organic solvent is acetone. In some embodiments, an organic solvent is ethanol. In some embodiments, an organic solvent is isopropanol. In some embodiments, an organic solvent is ethyl acetate. In some embodiments, an organic solvent is acetaldehyde. In some embodiments, an organic solvent is heptane. In some embodiments, an organic solvent is acetaldehyde. In some embodiments, an organic solvent is selected from methanol and dichloromethane.

In some embodiments, a composition comprises less than 10% by weight of an organic solvent selected from methanol and dichloromethane. It is understood that organic solvent content is determined by any method known to those of skill in the art, including chromatographic methods (e.g., gas chromatography). In some embodiments, a composition comprises less than 5% by weight of an organic solvent selected from methanol and dichloromethane. In some embodiments, a composition comprises less than 5% by weight of methanol. In some embodiments, a composition comprises less than 5% by weight of dichloromethane.

In some embodiments, a composition comprises less than or equal to 4% by weight of water. In some embodiments, a composition comprises less than or equal to 3% by weight of water. In some embodiments, a composition comprises less than or equal to 2% by weight of water. It is understood that water content is determined by any method known to those of skill in the art, including, for example, Karl Fisher analysis (e.g., coulometric Karl Fisher or volumetric Karl Fisher). In some embodiments, a composition comprises less than or equal to 1.5% by weight of water. In some embodiments, a composition comprises less than or equal to 1% by weight of water. In some embodiments, a composition comprises less than or equal to 0.5% by weight of water.

### Pharmaceutical Compositions Comprising Compound 1

In some embodiments, the present disclosure provides a pharmaceutical composition comprising, carbidopa, entacapone, and greater than 0.15% by weight of Compound 1:

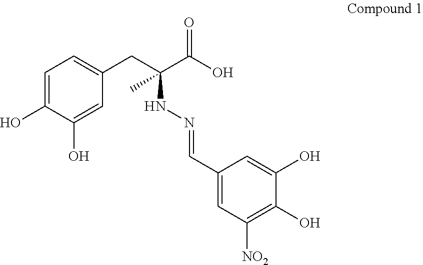 [SMILES: C[C@@](Cc1ccc(O)c(O)c1)(N/N=C/c1cc(O)c(O)c([N+](=O)[O-])c1)C(=O)O]

or a pharmaceutically acceptable salt thereof.

In some embodiments, carbidopa is carbidopa monohydrate.

In some embodiments, a pharmaceutical composition further comprises levodopa. In other words, in some embodiments, a pharmaceutical composition comprises carbidopa, entacapone, and greater than 0.15% by weight of Compound 1:

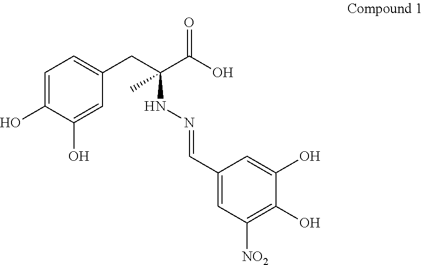 [SMILES: C[C@@](Cc1ccc(O)c(O)c1)(N/N=C/c1cc(O)c(O)c([N+](=O)[O-])c1)C(=O)O]

or a pharmaceutically acceptable salt thereof, and levodopa.

In some embodiments, carbidopa is carbidopa monohydrate.

In some embodiments, pharmaceutical compositions described herein are stored for a particular period of time before administration to a patient. In some embodiments, a pharmaceutical composition described herein is stable a particular temperatures and pressures for a particular period of time. For example after a particular period of time and/or at a particular temperature and/or humidity, in some embodiments, a pharmaceutical composition described herein is stable if the pharmaceutical composition comprises 1% or less by weight of an impurity. In some embodiments, a pharmaceutical composition is stable if the pharmaceutical composition comprises less than 0.75% or less by weight of an impurity. In some embodiments, a pharmaceutical composition is stable if the pharmaceutical composition comprises less than 0.50% or less by weight of an impurity. In some embodiments, a pharmaceutical composition is stable if the pharmaceutical composition comprises less than 0.40% or less by weight of an impurity. In some embodiments, a pharmaceutical composition is stable if the pharmaceutical composition comprises less than 0.25% or less by weight of an impurity. In some embodiments, a pharmaceutical composition is stable if the pharmaceutical composition comprises less than 0.15% or less by weight of an impurity. In some embodiments, a pharmaceutical composition is stable if the pharmaceutical composition comprises less than 0.10% or less by weight of an impurity. In some embodiments, a pharmaceutical composition is stable if the pharmaceutical composition is substantially free of any impurities.

In some embodiments, a pharmaceutical composition is stable for about 16 weeks or longer when stored at about 5° C. In some embodiments, a pharmaceutical composition is stable for about 20 weeks or longer when stored at about 5° C. In some embodiments, a pharmaceutical composition is stable for about 25 weeks or longer when stored at about 5° C. In some embodiments, a pharmaceutical composition is stable for about 30 weeks or longer when stored at about 5° C. In some embodiments, a pharmaceutical composition is stable for about 32 weeks or longer when stored at about 5° C.

In some embodiments, a pharmaceutical composition is stable for about 36 weeks or longer when stored at about 5° C. In some embodiments, a pharmaceutical composition is stable for about 40 weeks or longer when stored at about 5° C. In some embodiments, a pharmaceutical composition is stable for about 44 weeks or longer when stored at about 5° C. In some embodiments, a pharmaceutical composition is stable for about 48 weeks or longer when stored at about 5° C. In some embodiments, a pharmaceutical composition is stable for about 52 weeks or longer when stored at about 5° C.

In some embodiments, a pharmaceutical composition is stable for about 16 weeks or longer when stored at about 25° C. In some embodiments, a pharmaceutical composition is stable for about 20 weeks or longer when stored at about 25° C. In some embodiments, a pharmaceutical composition is stable for about 25 weeks or longer when stored at about 25° C. In some embodiments, a pharmaceutical composition is stable for about 30 weeks or longer when stored at about 25° C. In some embodiments, a pharmaceutical composition is stable for about 32 weeks or longer when stored at about 25° C. In some embodiments, a pharmaceutical composition is stable for about 36 weeks or longer when stored at about 25° C. In some embodiments, a pharmaceutical composition is stable for about 40 weeks or longer when stored at about 25° C. In some embodiments, a pharmaceutical composition is stable for about 44 weeks or longer when stored at about 25° C. In some embodiments, a pharmaceutical composition is stable for about 48 weeks or longer when stored at about 25° C. In some embodiments, a pharmaceutical composition is stable for about 52 weeks or longer when stored at about 25° C.

In some embodiments, a pharmaceutical composition comprises less than 2% by weight of water after storage for 16 weeks or longer at about 5° C. In some embodiments, a pharmaceutical composition comprises less than 2% by weight of water after storage for 16 weeks or longer at about 25° C. In some embodiments, a pharmaceutical composition comprises less than 3% by weight of water after storage for 26 weeks or longer at about 5° C. In some embodiments, a pharmaceutical composition comprises less than 3% by weight of water after storage for 26 weeks or longer at about 25° C. In some embodiments, a pharmaceutical composition comprises less than 4% by weight of water after storage for 52 weeks or longer at about 5° C. In some embodiments, a pharmaceutical composition comprises less than 4% by weight of water after storage for 52 weeks or longer at about 25° C. In some embodiments, a pharmaceutical composition comprises less than 4.5% by weight of water after storage for 52 weeks or longer at about 25° C.

In some embodiments, a pharmaceutical composition is stable for about 16 weeks or longer when stored at 5° C.±3° C. In some embodiments, a pharmaceutical composition is stable for about 20 weeks or longer when stored at 5° C.±3° C. In some embodiments, a pharmaceutical composition is stable for about 25 weeks or longer when stored at 5° C.±3° C. In some embodiments, a pharmaceutical composition is stable for about 30 weeks or longer when stored at 5° C.±3° C. In some embodiments, a pharmaceutical composition is stable for about 32 weeks or longer when stored at 5° C.±3° C.

In some embodiments, a pharmaceutical composition is stable for about 36 weeks or longer when stored at 5° C.±3° C. In some embodiments, a pharmaceutical composition is stable for about 40 weeks or longer when stored at 5° C.±3° C. In some embodiments, a pharmaceutical composition is stable for about 44 weeks or longer when stored at 5° C.±3° C. In some embodiments, a pharmaceutical composition is stable for about 48 weeks or longer when stored at 5° C.±3° C. In some embodiments, a pharmaceutical composition is stable for about 52 weeks or longer when stored at 5° C.±3° C.

In some embodiments, a pharmaceutical composition comprises less than 2% by weight of water after storage for 16 weeks or longer at 5° C.±3° C. In some embodiments, a pharmaceutical composition comprises less than 2% by weight of water after storage for 16 weeks or longer at 25° C.±2° C. In some embodiments, a pharmaceutical composition comprises less than 3% by weight of water after storage for 26 weeks or longer at 5° C.±3° C. In some embodiments, a pharmaceutical composition comprises less than 3% by weight of water after storage for 26 weeks or longer at 25° C.±2° C. In some embodiments, a pharmaceutical composition comprises less than 4% by weight of water after storage for 52 weeks or longer at 5° C.±3° C. In some embodiments, a pharmaceutical composition comprises less than 4% by weight of water after storage for 52 weeks or longer at 25° C.±2° C. In some embodiments, a pharmaceutical composition comprises less than 4.5% by weight of water after storage for 52 weeks or longer at 25° C.±2° C.

In some embodiments, a pharmaceutical composition is stable for 16 hours or longer at physiological temperature (e.g., 37° C.). In some embodiments, a pharmaceutical composition is stable for 24 hours or longer at physiological temperature (e.g., 37° C.). In some embodiments, a pharmaceutical composition is stable for 32 hours or longer at physiological temperature (e.g., 37° C.). In some embodiments, a pharmaceutical composition is stable for 48 hours or longer at physiological temperature (e.g., 37° C.).

Previous formulations comprising carbidopa and entacapone suffered from poor stability at room or physiological temperatures, and often needed to be discarded after 16 weeks when stored at 5° C.

In some embodiments, a pharmaceutical composition comprises about 0.1-2% by weight carbidopa. In some embodiments, a pharmaceutical composition comprises about 0.1-1% by weight of carbidopa. In some embodiments, a pharmaceutical composition comprises about 0.1, 0.2, 0.3, 0.4, 0.5, 0.6, 0.7, 0.8, 0.9, or 1% by weight of carbidopa. In some embodiments, a pharmaceutical composition comprises about 0.5% by weight of carbidopa.

In some embodiments, a pharmaceutical composition comprises about 1-7.5% by weight of entacapone. In some embodiments, a pharmaceutical composition comprises about 1-5% by weight of entacapone. In some embodiments, a pharmaceutical composition comprises about 1-3% by weight of entacapone. In some embodiments, a pharmaceutical composition comprises about 1, 1.5, 2, 2.5, or 3% by weight of entacapone. In some embodiments, a pharmaceutical composition comprises about 3% by weight of entacapone.

In some embodiments, a pharmaceutical composition comprises about 1-15% by weight of levodopa. In some embodiments, a pharmaceutical composition comprises about 1-10% by weight of levodopa. In some embodiments, a pharmaceutical composition comprises about 1-5% by weight of levodopa. In some embodiments, a pharmaceutical composition comprises about 1, 1.5, 2, 2.5, 3, 3.5, 4, 4.5, or 5% by weight of levodopa. In some embodiments, a pharmaceutical composition comprises about 2% by weight of levodopa.

In some embodiments, a pharmaceutical composition comprises greater than 0.15% by weight of Compound 1. In some embodiments, a pharmaceutical composition comprises about 0.15-1.5% by weight of Compound 1. In some embodiments, a pharmaceutical composition comprises about 0.15-1% by weight of Compound 1. In some embodiments, a pharmaceutical composition comprises about 0.15-0.8% by weight of Compound 1. In some embodiments, a pharmaceutical composition comprises about 0.15-0.5% by weight of Compound 1. In some embodiments, a pharmaceutical composition comprises about 0.15-0.4% by weight of Compound 1 In some embodiments, a pharmaceutical composition comprises about 0.15-0.2% by weight of Compound 1.

In some embodiments, a pharmaceutical composition comprises about 1-15% by weight of levodopa, about 0.1-2% by weight of carbidopa, and about 1-7.5% by weight of entacapone. In some embodiments, a pharmaceutical composition comprises about 2% by weight of entacapone, about 0.5% by weight of carbidopa, and about 2% by weight of entacapone.

In some embodiments, a pharmaceutical composition comprises about 12.5 mg to about 50 mg of carbidopa. In some embodiments, a pharmaceutical composition comprises about 12.5 mg, 18.75 mg, 25 mg, 31.25 mg, 37.5 mg, or 50 mg of carbidopa. In some embodiments, a pharmaceutical composition comprises about 12.5 mg of carbidopa. In some embodiments, a pharmaceutical composition comprises about 18.75 mg of carbidopa. In some embodiments, a pharmaceutical composition comprises about 25 mg of carbidopa. In some embodiments, a pharmaceutical composition comprises about 31.25 mg of carbidopa. In some embodiments, a pharmaceutical composition comprises about 37.5 mg of carbidopa. In some embodiments, a pharmaceutical composition comprises about 50 mg of carbidopa.

In some embodiments, a pharmaceutical composition comprises about 50 mg to about 200 mg of levodopa. In some embodiments, a pharmaceutical composition comprises about 50 mg, about 75 mg, about 100 mg, about 125 mg, about 150 mg, or about 200 mg of levodopa. In some embodiments, a pharmaceutical composition comprises about 50 mg of levodopa. In some embodiments, a pharmaceutical composition comprises about 25 mg of levodopa. In some embodiments, a pharmaceutical composition comprises about 100 mg of levodopa. In some embodiments, a pharmaceutical composition comprises about 125 mg of levodopa. In some embodiments, a pharmaceutical composition comprises about 150 mg of levodopa. In some embodiments, a pharmaceutical composition comprises about 200 mg of levodopa.

In some embodiments, a pharmaceutical composition comprises about 200 mg of entacapone.

In some embodiments, a pharmaceutical composition comprises a weight ratio of carbidopa to levodopa of about 1:4.

In some embodiments, a pharmaceutical composition comprises about 12.5 mg of carbidopa, about 50 mg of levodopa, and about 200 mg of entacapone. In some embodiments, a pharmaceutical composition comprises about 18.75 mg of carbidopa, about 75 mg of levodopa, and about 200 mg of entacapone. In some embodiments, a pharmaceutical composition comprises about 25 mg of carbidopa, about 100 mg of levodopa, and about 200 mg of entacapone. In some embodiments, a pharmaceutical composition comprises about 31.25 mg of carbidopa, about 125 mg of levodopa, and about 200 mg of entacapone. In some embodiments, a pharmaceutical composition comprises about 37.5 mg of carbidopa, about 150 mg of levodopa, and about 200 mg of entacapone. In some embodiments, a pharmaceutical composition comprises about 50 mg of carbidopa, about 200 mg of levodopa, and about 200 mg of entacapone.

In some embodiments, a pharmaceutical composition described herein further comprises one or more pharmaceutical carrier, adjuvant, or vehicle. In certain embodiments, a composition described herein is formulated for administration to a patient in need of such composition. In some embodiments, a composition described herein is formulated for oral administration to a patient. In some embodiments, a composition described herein is formulated for intragastric (e.g., intestinal) administration to a patient.

Compositions according to methods of the present disclosure are administered using any amount and any route of administration effective for treating or lessening the severity of a disorder provided herein. The exact amount required will vary from subject to subject, depending on the species, age, and general condition of the subject, the severity of the infection, the particular agent, its mode of administration, and the like. Compositions described herein are preferably formulated in unit dosage form for ease of administration and uniformity of dosage.

Compositions of the present disclosure may be administered orally, parenterally, by inhalation spray, topically, rectally, nasally, buccally, vaginally, enterically by direct administration to the intestine (i.e., intragastric administration) intraperitoneally, intracisternally or via an implanted reservoir. In some embodiments, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose, sodium carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In some embodiments, provided pharmaceutically acceptable compositions are formulated for oral administration. Such formulations may be administered with or without food. In some embodiments, pharmaceutically acceptable compositions described herein are administered without food. In other embodiments, pharmaceutically acceptable compositions described herein are administered with food. Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, sodium carboxymethylcellulose, alginates, gelatin, polyvinylpyrrolidinone, carbomer, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Alternatively, pharmaceutically acceptable compositions described herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Dosage forms for topical or transdermal administration of a compound disclosed herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Intragastric forms for administration directly to the intestine of the patient include gels and the like. The active components are admixed with a pharmaceutically acceptable carrier, along with any other preservatives or buffers that may be required. For example, in some embodiments, a pharmaceutical composition described herein further comprises one or more pharmaceutical carriers. In some embodiments, a pharmaceutical carrier is a polysaccharide. In some embodiments, a polysaccharide is a cellulose derivative. In some embodiments, a polysaccharide is methylcellulose, ethylcellulose, or carboxymethyl cellulose or sodium carboxymethylcellulose In some embodiments, a polysaccharide is methylcellulose. In some embodiments, a polysaccharide is ethylcellulose. In some embodiments, a polysaccharide is or carboxymethyl cellulose.

In some embodiments, a pharmaceutical composition comprises about 2-3% by weight of a polysaccharide. In some embodiments, a pharmaceutical composition comprises about 2-3% by weight of a cellulose derivative. In some embodiments, a pharmaceutical composition comprises about 2-3% by weight of methylcellulose, ethylcellulose, or carboxymethyl cellulose or sodium carboxymethyl cellulose. In some embodiments, a pharmaceutical composition comprises about 2-3% by weight of methylcellulose. In some embodiments, a pharmaceutical composition comprises about 2-3% by weight of ethylcellulose. In some embodiments, a pharmaceutical composition comprises about 2-3% by weight of carboxymethyl cellulose.

In some embodiments, a pharmaceutical composition is in the form of a gel. In some embodiments, a pH of a gel described herein is less than about 5.7. In some embodiments, a pH of a gel described herein is about 4.5-5.7. In some embodiments, a pH of a gel described herein is about 4.5-5.5. In some embodiments, a pH of a gel described herein is about 5.0.

In some embodiments, a pharmaceutical composition is a gel, and a gel has a viscosity of about 3000-5000 CPS, as measured by Brookfield DVII TRV at 50 RPM and at the incubation temperature of 25° C. using small sample adapter, spindle SC4-28, and solid shaft. In some embodiments, a pharmaceutical composition is a gel, and a gel has a viscosity of about 3500-4500 CPS, as measured by Brookfield DVII TRV at 50 RPM and at the incubation temperature of 25° C. using small sample adapter, spindle SC4-28, and solid shaft.

### Methods of Treating Neurodegenerative Disorders

In some embodiments, the present disclosure provides methods for treating diseases, disorders, and conditions using pharmaceutical compositions described herein. In some embodiments, the present disclosure provides a method of treating a neurological disease by administering to a patient a pharmaceutical composition described herein.

For example, in some embodiments, the present disclosure provides a method of treating a neurological disease comprising a pharmaceutical composition comprising carbidopa, entacapone, and greater than 0.15% by weight of Compound 1:

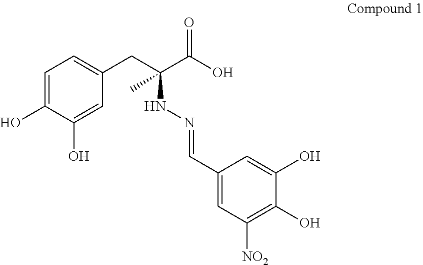 [SMILES: C[C@@](Cc1ccc(O)c(O)c1)(N/N=C/c1cc(O)c(O)c([N+](=O)[O-])c1)C(=O)O]

or a pharmaceutically acceptable salt thereof.

In some embodiments, a neurological disorder is Parkinson's Disease. In some embodiments, Parkinson's Disease is Advanced Parkinson's Disease.

In some embodiments, a pharmaceutical composition is administered to a patient continuously over about 16 hours. In some embodiments, about 25% of a daily dose of a pharmaceutical composition described herein is administered to the patient in the morning. In some embodiments, In some embodiments, a pharmaceutical composition is administered to the patient over 16 hours, where about 25% of a daily dose of a pharmaceutical composition is administered to the patient in the morning, followed by continuous administration of a pharmaceutical composition to the patient throughout the remainder of the 16 hour dose time.

In some embodiments, a pharmaceutical composition is administered to the patient once daily.

In some embodiments, a pharmaceutical composition is administered directly to the intestine of a patient. In some embodiments, a pharmaceutical composition is administered by a PEG-J tube.

In some embodiments, a pharmaceutical composition is the form of an oral dosage form. In some embodiments, an oral dosage form is a capsule or tablet. In some embodiments, an oral dosage form is a capsule. In some embodiments, an oral dosage form is a tablet.

In some embodiments, a pharmaceutical composition is in the form of an oral dosage form, and the oral dosage form is administered to the patient such that the total daily dose of levodopa administered to the patient is less than or equal to 1200 mg.

In some embodiments, the present disclosure provides use of a pharmaceutical composition described herein for the treatment of a neurological disorder also described herein.

### Methods of Qualifying a Composition Comprising Compound 1

The present disclosure further provides, among other things, methods of qualifying or assessing for quality a pharmaceutical composition. In some embodiments, the present disclosure provides a method of characterizing the purity of a pharmaceutical composition comprising levodopa, carbidopa, and entacapone, the method comprising determining a quantity of Compound 1:

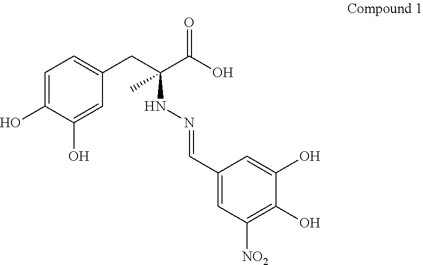 [SMILES: C[C@@](Cc1ccc(O)c(O)c1)(N/N=C/c1cc(O)c(O)c([N+](=O)[O-])c1)C(=O)O]

in the pharmaceutical composition by comparison of a sample of the pharmaceutical composition to a reference standard, wherein the reference standard comprises a known quantity of Compound 1.

In some embodiments, a sample of a pharmaceutical composition is classified as being high purity if the pharmaceutical composition comprises less than about 0.15% by weight of Compound 1. In some embodiments, a sample of a pharmaceutical composition is classified as being middle purity if the pharmaceutical composition comprises about 0.15-1% by weight of Compound 1. In some embodiments, a sample of a pharmaceutical composition is classified as being high purity if the pharmaceutical composition comprises about 0.5% or less by weight of Compound 1. In some embodiments, a sample of a pharmaceutical composition is classified as being high purity if the pharmaceutical composition comprises about 0.4% or less by weight of Compound 1. In some embodiments, a sample of a pharmaceutical composition is classified as being low purity if the pharmaceutical composition comprises about 1% or less by weight of Compound 1.

In some embodiments, a reference standard comprises a composition comprising 95% or greater by weight of Compound 1. In some embodiments, a reference standard comprises a composition comprising about 95, 96, 97, 98, 99, or 99.9% by weight of Compound 1. In some embodiments, a reference standard comprises a composition consisting essentially of Compound 1.

In some embodiments, the present disclosure provides a method of using a reference standard comprising a known quantity of Compound 1:

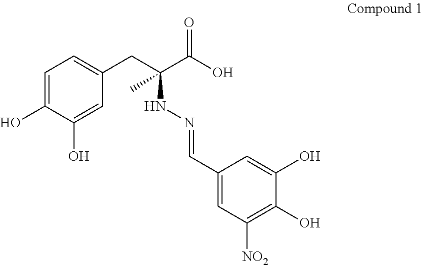 [SMILES: C[C@@](Cc1ccc(O)c(O)c1)(N/N=C/c1cc(O)c(O)c([N+](=O)[O-])c1)C(=O)O]

to determine a level of purity of a pharmaceutical composition, the method comprising comparing a quantity of Compound 1 in the reference standard to a quantity of Compound 1 in the pharmaceutical composition.

In some embodiments, a quantity of Compound 1 in a pharmaceutical composition is assessed by high pressure liquid chromatography (HPLC) using a Waters SunFire C18 Column, 150×4.6 mm, 3.5 um, UV/Vis detector, a mobile phase A that is 0.06% triflouroacetic acid (TFA) in water, and a mobile phase B that is 0.04% TFA in (80:20 MeOH;H₂O).

### Methods of Making Compound 1

In some embodiments, the present disclosure provides a method of manufacturing a composition comprising Compound 1

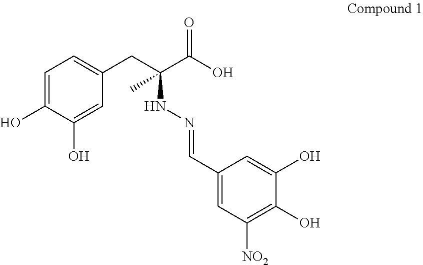 [SMILES: C[C@@](Cc1ccc(O)c(O)c1)(N/N=C/c1cc(O)c(O)c([N+](=O)[O-])c1)C(=O)O]

comprising contacting Compound 2

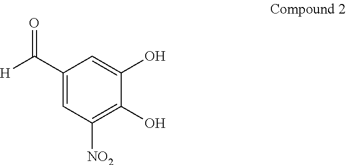 [SMILES: O=Cc1cc(O)c(O)c([N+](=O)[O-])c1]

with Compound 3

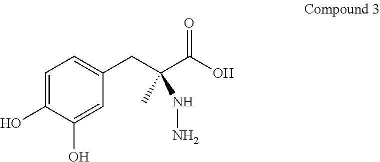 [SMILES: C[C@@](Cc1ccc(O)c(O)c1)(NN)C(=O)O]

in the presence of an organic solvent and a sulfate salt.

In some embodiments, a sulfate salt is sodium sulfate, potassium sulfate, magnesium sulfate, calcium sulfate, or ammonium sulfate. In some embodiments, a sulfate salt is sodium sulfate. In some embodiments, a sulfate salt is potassium sulfate. In some embodiments, a sulfate salt is magnesium sulfate. In some embodiments, a sulfate salt is calcium sulfate. In some embodiments, a sulfate salt is ammonium sulfate.

In some embodiments, an organic solvent is methanol, ethanol, propanol, isopropanol, butanol or ethyl acetate. In some embodiments, an organic solvent is methanol, ethanol, propanol, isopropanol, or butanol. In some embodiments, an organic solvent is methanol. In some embodiments, an organic solvent is ethanol. In some embodiments, an organic solvent is propanol. In some embodiments, an organic solvent is isopropanol. In some embodiments, an organic solvent is butanol. In some embodiments, an organic solvent is ethyl acetate.

In some embodiments, a method of manufacturing Compound 1 further comprises suspending Compound 1 in an organic solvent, and filtering a solid form of Compound 1, to thereby provide a composition comprising Compound 1 that is greater than 90% by weight free from impurities (i.e., a composition comprising Compound 1 has less than 10% by weight of any impurities). In some embodiments, a method of manufacturing described herein provides Compound 1 that is greater than 95% by weight free from impurities. In some embodiments, a method of manufacturing described herein provides Compound 1 that is greater than 99% by weight free from impurities. In some embodiments, a method of manufacturing described herein provides Compound 1 that is greater than 99.9% by weight free from impurities. In some embodiments, a method of manufacturing described herein provides Compound 1 that is substantially free from impurities.

### EXEMPLARY EMBODIMENTS

The embodiments of the disclosure described above are intended to be merely exemplary, numerous variations and modifications will be apparent to those skilled in the art. All such variations and modifications are intended to be within the scope of the present invention as defined in any appended claims.
1. A composition comprising greater than about 0.15% by weight of Compound 1:

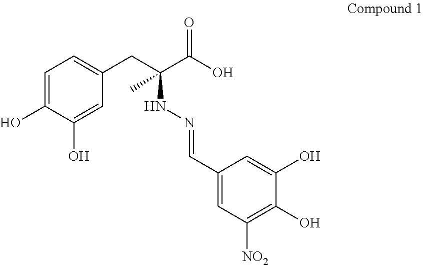 [SMILES: C[C@@](Cc1ccc(O)c(O)c1)(N/N=C/c1cc(O)c(O)c([N+](=O)[O-])c1)C(=O)O]

or a pharmaceutically acceptable salt thereof.
2. The composition of Embodiment 1, comprising greater than or equal to about 50% by weight of Compound 1.
3. The composition of Embodiments 1 or 2, comprising greater than or equal to about 75% by weight of Compound 1.
4. The composition of any one of Embodiments 1-3, comprising greater than or equal to about 85% by weight of Compound 1.
5. The composition of any one of Embodiments 1-4, comprising greater than or equal to about 90% by weight of Compound 1.
6. The composition of any one of Embodiments 1-5, comprising greater than or equal to about 95% by weight of Compound 1.
7. The composition of any one of Embodiments 1-6, wherein the composition comprises less than 10% by weight of an organic solvent.
8. The composition of any one of Embodiments 1-7, wherein the composition comprises less than 5% by weight of an organic solvent.
9 The composition of Embodiments 7 or 8, wherein the organic solvent is selected from the group consisting of methanol, dichloromethane, acetone, ethanol, isopropanol, ethyl acetate, acetaldehyde, heptane, and acetaldehyde.
10. The composition of any one of Embodiments 1-9, wherein the composition comprises less than or equal to about 2% by weight of water.
11. The composition of any one of Embodiments 1-10, wherein the composition comprises less than or equal to about 1.5% by weight of water.
12. The composition of Embodiments 10 or 11, wherein a weight of water is determined by Karl Fisher analysis.
13. A pharmaceutical composition comprising, carbidopa, entacapone, and greater than 0.15% by weight of Compound 1:

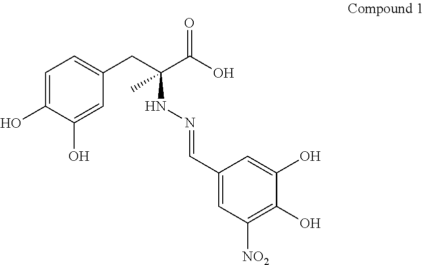 [SMILES: C[C@@](Cc1ccc(O)c(O)c1)(N/N=C/c1cc(O)c(O)c([N+](=O)[O-])c1)C(=O)O]

or a pharmaceutically acceptable salt thereof.
14. The pharmaceutical composition of Embodiment 13, further comprising levodopa.
15. The pharmaceutical composition of Embodiments 13 or 14, characterized in that the pharmaceutical composition is stable for 16 weeks or longer when stored at about 5° C.
16. The pharmaceutical composition of any one of Embodiments 13-15, characterized in that the pharmaceutical composition is stable for 16 hours or longer at physiological temperature (e.g., 37° C.).
17. The pharmaceutical composition of any one of Embodiments 14-16, wherein the pharmaceutical composition comprises:
   about 1-15% by weight of levodopa;
   about 0.1-2% by weight of carbidopa; and
   about 1-7.5% by weight of entacapone.
18. The pharmaceutical composition of any one of Embodiments 14-17, wherein the pharmaceutical composition comprises:
   about 2% by weight of levodopa;
   about 0.5% by weight of carbidopa; and
   about 2% by weight of entacapone.
19. The pharmaceutical composition of any one of Embodiments 13-18, further comprising one or more pharmaceutical carriers.
20. The pharmaceutical composition of Embodiment 19, wherein the one or more pharmaceutical carriers is a polysaccharide.
21. The pharmaceutical composition of Embodiment 20, wherein the polysaccharide is sodium carboxymethyl cellulose.
22 The pharmaceutical composition of Embodiment 20, wherein the pharmaceutical composition comprises about 2-3% by weight of the sodium carboxymethyl cellulose.
23. The pharmaceutical composition of any one of Embodiments 13-22, wherein the pharmaceutical composition is in the form of a gel.
24. The pharmaceutical composition of Embodiment 23, wherein the pH of the gel is less than about 5.7.
25. The pharmaceutical composition of Embodiments 23 or 24, wherein the pH of the gel is about 4.5 to about 5.5.
26. The pharmaceutical composition of any one of Embodiments 23-25, wherein the pH of the gel is about 5.0.
27 The pharmaceutical composition of any one of Embodiments 23-26, wherein the viscosity of the gel is from about 3000 to about 5000 CPS.
28. The pharmaceutical composition of any one of Embodiments 23-27, wherein the viscosity of the gel is from about 3500 to about 4500 CPS.
29. The pharmaceutical composition of any one of Embodiments 23-28, wherein the gel is formulated to be administered intestinally.
30 The pharmaceutical composition of any one of Embodiments 13-22, wherein the pharmaceutical composition is in the form of an oral dosage form.
31. A method of treating Parkinson's Disease (PD) in a patient comprising administering to the subject a pharmaceutical composition of any one of Embodiments 13-29.
32. The method of Embodiment 31, wherein the Parkinson's Disease is Advanced Parkinson's Disease.
33. The method of Embodiments 31 or 32, wherein the pharmaceutical composition is administered to the patient over a 16 hour course.
34. The method of any one of Embodiments 31-33, wherein about 25% of a total daily dose of the pharmaceutical composition is administered to the patient in the morning.
35 The method of any one of Embodiments 31-34, wherein the pharmaceutical composition is administered to the patient once daily.
36 The method of any one of Embodiments 31-35, wherein the pharmaceutical composition is administered directly to the intestine.
37. The method of Embodiment 36, wherein the pharmaceutical composition is administered by a PEG-J tube.
38. A method of treating Parkinson's Disease (PD) in a patient comprising administering to the subject a pharmaceutical composition of Embodiment 30.
39. The method of Embodiment 38, wherein the Parkinson's Disease is Advanced Parkinson's Disease.
40. A method of manufacturing a composition comprising Compound 1

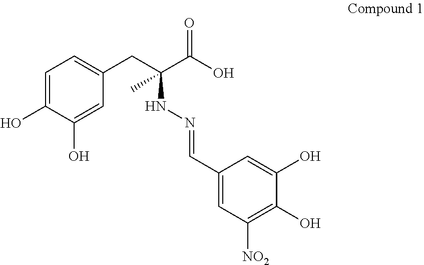 [SMILES: C[C@@](Cc1ccc(O)c(O)c1)(N/N=C/c1cc(O)c(O)c([N+](=O)[O-])c1)C(=O)O]

comprising contacting Compound 2

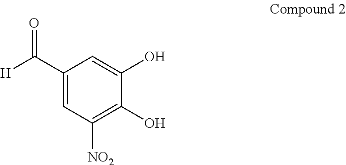 [SMILES: O=Cc1cc(O)c(O)c([N+](=O)[O-])c1]

with Compound 3

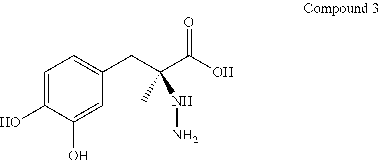 [SMILES: C[C@@](Cc1ccc(O)c(O)c1)(NN)C(=O)O]

in the presence of an organic solvent and a sulfate salt.
41. The method of Embodiment 40, wherein the sulfate salt is sodium sulfate, potassium sulfate, magnesium sulfate, calcium sulfate, or ammonium sulfate.
42. The method of Embodiments 40 or 41, wherein the sulfate salt is sodium sulfate.
43. The method of any one of Embodiments 40-42, wherein the organic solvent is methanol, ethanol, propanol, isopropanol, or butanol.
44. The method of Embodiment 43, wherein the organic solvent is methanol.
45. The method of any one of Embodiments 40-44, further comprising suspending Compound 1 in an organic solvent, and filtering a solid form of Compound 1, to thereby provide a composition comprising Compound 1 that is greater than 90% by weight free from impurities.
46. The method of Embodiment 45, wherein the composition comprising Compound 1 is greater than 95% by weight free from impurities.
47. The method of Embodiment 46, wherein the composition comprising Compound 1 is greater than 99% by weight free from impurities.
48. The method of Embodiment 47, wherein the composition comprising Compound 1 is substantially free from impurities.
49. The method of any one of Embodiments 40-48, wherein the organic solvent is dichloromethane.
50. A method of characterizing the purity of a pharmaceutical composition comprising levodopa, carbidopa, and entacapone, the method comprising determining a quantity of Compound 1:

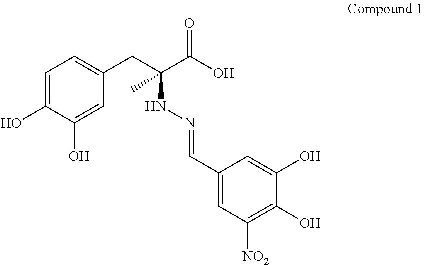 [SMILES: C[C@@](Cc1ccc(O)c(O)c1)(N/N=C/c1cc(O)c(O)c([N+](=O)[O-])c1)C(=O)O]

in the pharmaceutical composition by comparison of a sample of the pharmaceutical composition to a reference standard, wherein the reference standard comprises a known quantity of Compound 1.
51. The method of Embodiment 50, wherein the pharmaceutical composition is characterized as being high purity if the pharmaceutical composition comprises less than about 0.15% by weight of Compound 1.
52. The method of Embodiments 50 or 51, wherein the reference standard is a composition comprising 95% or greater by weight of Compound 1.
53. The method of any one of Embodiments 50-52, wherein the purity of the pharmaceutical composition is assessed by high pressure liquid chromatography (HPLC) using a Waters SunFire C18 Column, 150 λ4.6 mm, 3.5 um, UV/Vis detector, a mobile phase A that is 0.06% triflouroacetic acid (TFA) in water, and a mobile phase B that is 0.04% TFA in (80:20 MeOH;H₂O).
54. A method of using a reference standard comprising a known quantity of Compound 1:

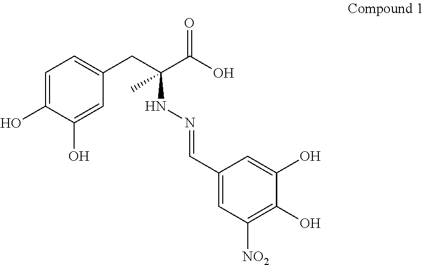 [SMILES: C[C@@](Cc1ccc(O)c(O)c1)(N/N=C/c1cc(O)c(O)c([N+](=O)[O-])c1)C(=O)O]

to determine a level of purity of a pharmaceutical composition, the method comprising comparing a quantity of Compound 1 in the reference standard to a quantity of Compound 1 in the pharmaceutical composition.
55. The method of Embodiment 54, wherein the purity of the pharmaceutical composition is assessed by high pressure liquid chromatography (HPLC) using a Waters SunFire C18 Column, 150×4.6 mm, 3.5 um, UV/Vis detector, a mobile phase A that is 0.06% triflouroacetic acid (TFA) in water, and a mobile phase B that is 0.04% TFA in (80:20 MeOH;H₂O).

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

### Example 1—Synthesis of Compound 1

The present example provides a synthesis of Compound 1.

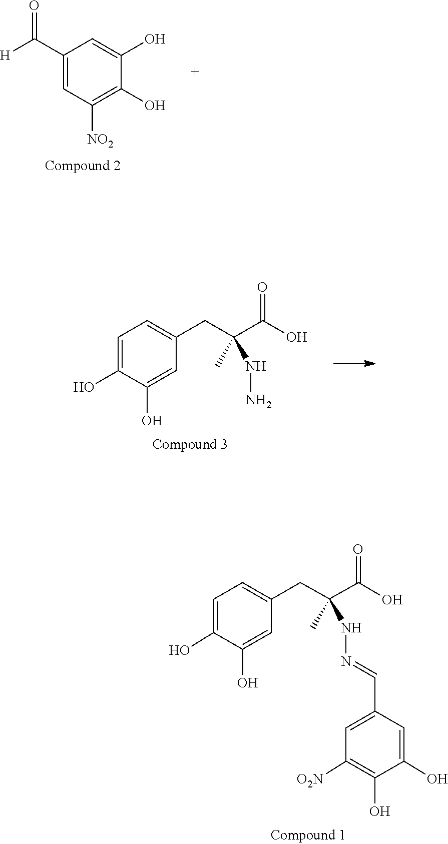 [SMILES: O=Cc1cc(O)c(O)c([N+](=O)[O-])c1 ; C[C@@](Cc1ccc(O)c(O)c1)(NN)C(=O)O ; C[C@@](Cc1ccc(O)c(O)c1)(N/N=C/c1cc(O)c(O)c([N+](=O)[O-])c1)C(=O)O]

77 g of Compound 2 (purchased from Combi-Blocks, CAS Number 116313-85-0) (1 eq.) and 100 g Compound 3 (purchased from Combi-Blocks, CAS number 28860-95-9) (1.05 eq.) was stirred at room temperature in a flask with Na₂SO₄ (1.5 w/w) and methanol (MeOH) for 1 hour. Sodium Sulfate was purchased from Caledon Laboratories Limited, CAS number 7757-82-6. Methanol (MeOH) was purchased from Caledon Laboratories Limited, CAS number 67-56-1. The resulting mixture was filtered and dried under vacuum to provide crude product.

The crude product was slurried with dichloromethane (DCM) for 18 hours. Dichloromethane (DCM) was purchased from Caledon Laboratories Limited, CAS number: 1975-09-02. The fine solid product was collected, and remaining solids were ground by mortal pestle, followed by re-suspension and stirring in DCM overnight. The fine solids were collected and all fine solids were combined, suspended in DCM, and stirred overnight. The resulting mixture was dried in vacuum oven to provide Compound 1.

### Example 2—Analysis of Compound 1

Compound 1 purity was determined by HPLC at two wavelengths 220 nm and 280 nm. Identification of Compound 1 was carried out by IR and NMR methods (¹H NMR, ¹³C NMR). The residual solvents methanol and dichloromethane (DCM) were determined by gas chromatography (GC). Residual water content was determined by Karl Fisher analysis. Thermogravimetric analysis (TGA) of Compound 1 was also carried out with hold time of 10 minutes at final temperature of 105° C.

The potency of the a composition comprising Compound 1 corrected with solvent, and residual water was calculated as per Alphora SOP LC038 ‘Standard Qualification in the QC/AS Laboratory’ using the following equation:

$$Potency = \frac{E 920 Total purity ( \% \frac{a}{a} ) \times ( 100 \% - \% water - \% solvents )}{100}$$

Details of the analysis are described below:

### 1. Materials and Equipment:

Column: Waters SunFire C18 Column, 150×4.6 mm, 3.5 um
HPLC Pump: Quaternary or Binary Pump
Detection: UV or Diode Array Detector
Integrator or computer-based data system.
Autosampler equipped with a sample cooling system.
Calibrated analytical balance.
Class A volumetric flasks and pipettes
HPLC vials and caps

### 2. Reagents and Standards:

Methanol (MeOH) HPLC grade or equivalent
Water (H₂O): MilliQ purified or equivalent
Trifluoroacetic Acid (TFA): HPLC grade or equivalent
Equivalent or higher grades may be used

### 3. Solutions

Volumes may be scaled to suit the needs of the analysis

### 3.1. Mobile Phase a (0.06% TFA in Water):

Transfer 0.6 ml of TFA to a container with 1 L H2O, mix well. Degas with sonication under vacuum.

### 3.2. Mobile Phase B: (0.04% TFA in (80:20 MeOH:H₂O).

Transfer 0.4 mL of TFA to a container with 800 mL of MeOH and 200 mL of H₂O in 1 L HPLC reservoir. Mix well. Degas with sonication under vacuum.

### 3.3 Diluent: 80:20 MeOH/H2O.

Transfer 160 mL of MeOH and 40 mL of H2O to a 200 mL HPLC reservoir. Mixed well and degas with sonication under vacuum.

### 3.4 Blank Solution: Diluent

ECA—Analytical Method—E920H₂—for standard qualification

### 4. Instrument Parameters

Detector: UV/Vis or Diode Array Detector
Wavelength: Signal A: 280 nm, Bandwidth: 4 nm, Reference: OFF
Signal B: 220 nm, Bandwidth: 4 nm, Reference: OFF
Column: SunFire C18 Column (150×4.6 mm, 3.5 um)
Mobile Phase A: 0.06% TFA in H2O
Mobile Phase B: 0.04% TFA in (MeOH:H2O, 80:20)

Gradient

| Time, mins | % A | % B |
|---|---|---|
| 0 | 100 | 0 |
| 8 | 85 | 15 |
| 25 | 0 | 100 |
| 25.1 | 100 | 0 |
| 30 | 100 | 0 |

Autosampler Temperature: 6° C.
Column Temperature: 40° C.
Flow Rate: 1.0 mL/min
Injection Volume: 5 μL
Run Time: 30 min

### 5. Purity Analysis

### 5.1 Sample Preparation (in Duplicate)

Accurately weigh approximately 10.0 mg of test sample into a 20 mL volumetric flask.
Dilute to volume with diluent and mix well. (0.5 mg/mL).

### 5.2 Injection Sequence

| Solution | # of Injections |
|---|---|
| Diluent Blank | 2 |
| Test sample prep 1 | 1 |
| Test sample prep 2 | 1 |

Note:
if necessary, additional Blank injections can be made until a stable and reproducible baseline is obtained. Additional Test Sample injections may be added as required.

### 5.3 Integration

Integrate all peaks≥0.05% a/a, not present in the blank solution.
ECA—Analytical Method—E920H₂—for standard qualification

### 6. Chromatographic System Suitability Requirements

### 6.1 Blank Solution Injection:

A stable and reproducible baseline should be observed. Blank solution injections may be repeated until this condition is met.

### 6.2 Peak Identification:

The chromatographic profiles of the replicate preparations must be comparable.

### 7. Calculations

### 7.1 Individual Impurities:

Report the percent impurity values to two decimal places for any impurity ≥0.05% a/a. Do not report any impurities <0.05% a/a. If only one value ≥0.05% a/a appears, report the value based on a single injection.
   7.2 Total Impurities (% a/a)
Total Impurities (% a/a)=sum of all impurities ≥0.05% a/a

### 8. Reporting

Report results at each wavelength, 220 nm and 280 nm
   8.1. For all impurities detected ≥0.05% a/a, report:
Amount (% a/a)
Relative retention time
Total Impurities: % a/a to one decimal place
Note: for determination of potency, use purity results from 280 nm

### 9. Typical Retention Times and Example Chromatograms:

### 9.1 Typical Retention Times:

| Component RT, | min | RRT |
|---|---|---|
| Compound 1 | 21.578 | 1.0 |

ECA—Analytical Method—E920H₂—for standard quality

### Example 3—Stability of Compositions Comprising Compound 1

The stability profile of Compound 1 (Eurofins CDMO Alphora Inc. code: E920) was being evaluated over a period of 6 months under Long Term storage condition of <−20° C. and 6 months under accelerated condition of 25±2° C./60%±5% RH; and at intermediate storage conditions of 5±3° C. Additional samples are stored at each storage conditions to cover the stability studies up to 12 months, after 6 months satisfactory stability results. Attributes being tested include: description of composition, purity by HPLC, identification by HPLC and water content by Karl Fischer (KF) Method. This study is conducted in compliance with ICH Q1A (R2) and Alphora Standard Operating Procedure SOP LC027.

The following test conditions described below are studied and reflect the long-term storage conditions of E920. The storage conditions also reflect the packaging configuration, storage and shipping conditions proposed at the time of material manufacture.

| Storage type | Condition | |
|---|---|---|
| Long-term conditions | −20° | C. |
| Intermediate condition | 5 ± 3° | C. |

| | |
|---|---|
| Accelerated conditions | 25 ± 2° C./60% ± 5% RH |

### Test Methods and Specification

The test attributes included in the release specification, and those included in stability testing are listed below.

Test Attributes Included in Release and Stability Specifications, with Justification

| | | Included in | | Limits and reporting | |
|---|---|---|---|---|---|
| Test | Test | release | Tested in | criteria for the stability | |
| attribute | Reference | specifications? | stability? | study | |
| Description | GEN-DESC | Yes | Yes | Report only | |
| | 1-P-QM- | | | | |
| | WI- | | | | |
| | 9101705 | | | | |
| Identification - | E920H2 | Yes | Yes | Conforms | |
| HPLC | 1-P-QM- | | | (% difference of retention | |
| | WI- | | | time vs standard NMT 5%) | |
| Impurities - | 9122883 | Yes | Yes | E920 | Report Only, |
| HPLC | | | | | % a/a |
| | | | | | (Target NLT |
| | | | | | 95% a/a) |
| | | | | Each individual | Report only, |
| | | | | impurity ≥0.05% | % a/a |
| | | | | a/a | |

| Water | KFC | Yes | Yes | Report only, % w/w |
|---|---|---|---|---|
| Content | 1-P-QM- | | | |
| (Karl | WI- | | | |
| Fischer) | 9101146 | | | |

### Test Schedule

The following table outlines the test schedule, by condition and time-point.

| | | Time | | | |
|---|---|---|---|---|---|
| Condition | Test | 0 | 1 M | 3 M | 6 M |
| Long term | Description | X | X | X | X |
| | Identification, purity - | X | X | X | X |
| | HPLC | | | | |
| | Water Content | X | X | X | X |
| Intermediate | Description | Store only | Store only | Store only | Store only |
| | Identification, purity - | Store only | Store only | Store only | Store only |
| | HPLC | | | | |
| | Water Content | Store only | Store only | Store only | Store only |
| Accelerated | Description | X | X | X | X |
| | Identification, purity - | X | X | X | X |
| | HPLC | | | | |
| | Water Content | X | X | X | X |

Results: Three-month stability are satisfactory and projected to be stable at −20° C. and 25° C./60% RH for longer time at least 6 months.

| | | −20° C. | | 25° C./60% RH | |
|---|---|---|---|---|---|
| Attributes | Initial | 1 M | 3 M | 1 M | 3 M |
| % Purity at 220 nm by HPLC | 95.4 | 95.8 | 94.3 | 95.7 | 93.9 |
| % Purity at 280 nm by HPLC | 96.1 | 96.0 | 95.2 | 96.2 | 94.9 |
| % residual water content by KF | 1.51 | 1.67 | 1.96 | 1.63 | 2.05 |

### Example 4—Stability of Compositions Comprising Compound 1

The stability profile of Compound 1 (Eurofins CDMO Alphora Inc. code: E920) was being evaluated over a period of up to 12 months under Long Term storage condition of <−20° C. and up to 12 months under accelerated condition of 25±2° C./60%±5% RH; and store only at intermediate storage conditions of 5±3° C. Attributes being tested include: description of composition, purity by HPLC, identification by HPLC and water content by Karl Fischer (KF) Method. This study is conducted in compliance with ICH Q1A (R2) and Alphora Standard Operating Procedure SOP LC027.

The following test conditions described below are studied and reflect the long-term storage conditions of E920. The storage conditions also reflect the packaging configuration, storage and shipping conditions proposed at the time of material manufacture.

| Storage type | Condition | |
|---|---|---|
| Long-term conditions | −20° | C. |
| Intermediate condition | 5 ± 3° | C. |

| | |
|---|---|
| Accelerated conditions | 25 ± 2° C./60% ± 5% RH |

### Test Methods and Specification

The test attributes included in the release specification, and those included in stability testing are listed below.

Test Attributes Included in Release and Stability Specifications, with Justification

| | | Included in | | Limits and reporting | |
|---|---|---|---|---|---|
| Test | Test | release | Tested in | criteria for the stability | |
| attribute | Reference | specifications? | stability? | study | |
| Description | GEN-DESC | Yes | Yes | Report only | |
| | 1-P-QM- | | | | |
| | WI- | | | | |
| | 9101705 | | | | |
| Identification - | E920H2 | Yes | Yes | Conforms | |
| HPLC | 1-P-QM- | | | (% difference of retention | |
| | WI- | | | time vs standard NMT 5%) | |
| Impurities - | 9122883 | Yes | Yes | E920 | Report Only, |
| HPLC | | | | | % a/a |
| | | | | | (Target NLT |
| | | | | | 95% a/a) |
| | | | | Each individual | Report only, |
| | | | | impurity ≥0.05% | % a/a |
| | | | | a/a | |
| Water | KFC | Yes | Yes | Report only, % w/w | |
| Content | 1-P-QM- | | | | |
| (Karl | WI- | | | | |
| Fischer) | 9101146 | | | | |

### Test Schedule

The following table outlines the test schedule, by condition and time-point.

| | | Time | | | | |
|---|---|---|---|---|---|---|
| Condition | Test | 0 | 1 M | 3 M | 6 M | 12 M |
| Long term | Description | X | X | X | X | X |
| | Identification, | X | X | X | X | X |
| | purity-HPLC | | | | | |
| | Water Content | X | X | X | X | X |
| Intermediate | Description | Store only | Store only | Store only | Store only | Store only |
| | Identification, | Store only | Store only | Store only | Store only | Store only |
| | purity-HPLC | | | | | |
| | Water Content | Store only | Store only | Store only | Store only | Store only |
| Accelerated | Description | X | X | X | X | X |
| | Identification, | X | X | X | X | X |
| | purity-HPLC | | | | | |
| | Water Content | X | X | X | X | X |

Results: Twelve-month stability are satisfactory and projected to be stable at −20° C. and 25° C./60% RH for longer time at least 12 months.

| | | −20° C. | | | |
|---|---|---|---|---|---|
| Attributes | Initial | 1 M | 3 M | 6 M | 12 M |
| % a/a Purity at 220 nm by HPLC | 95.4 | 95.8 | 94.3 | 96.5% | 95.9 |
| % a/a Purity at 280 nm by HPLC | 96.1 | 96.0 | 95.2 | 96.1 | 96.6 |
| % residual water content by KF | 1.51 | 1.67 | 1.96 | 3.35 | 4.08 |

| | | 25° C./60% RH | | | |
|---|---|---|---|---|---|
| Attributes | Initial | 1 M | 3 M | 6 M | 12 M |
| % a/a Purity at 220 nm by HPLC | 95.4 | 95.7 | 93.9 | 94.8 | 93.2 |
| % a/a Purity at 280 nm by HPLC | 96.1 | 96.2 | 94.9 | 95.3 | 95.1 |
| % residual water content by KF | 1.51 | 1.63 | 2.05 | 3.55 | 4.02 |

1. A composition comprising greater than about 0.15% by weight of Compound 1: 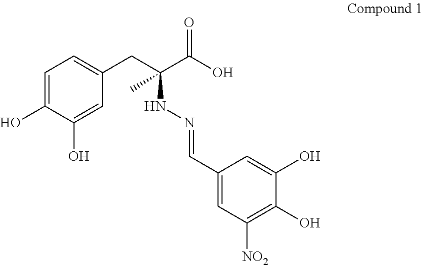 [SMILES: C[C@@](Cc1ccc(O)c(O)c1)(N/N=C/c1cc(O)c(O)c([N+](=O)[O-])c1)C(=O)O]
or a pharmaceutically acceptable salt thereof.

2. The composition of claim 1, comprising greater than or equal to about 50% by weight of Compound 1.

3. The composition of claim 1 or 2, comprising greater than or equal to about 75% by weight of Compound 1.

4. The composition of claim 1, comprising greater than or equal to about 85% by weight of Compound 1.

5. The composition of claim 1, comprising greater than or equal to about 90% by weight of Compound 1.

6. The composition of claim 1, comprising greater than or equal to about 95% by weight of Compound 1.

7. The composition of claim 1, wherein the composition comprises less than 10% by weight of an organic solvent.

8. The composition of claim 1, wherein the composition comprises less than 5% by weight of an organic solvent.

9. The composition of claim 7 or 8, wherein the organic solvent is selected from the group consisting of methanol, dichloromethane, acetone, ethanol, isopropanol, ethyl acetate, acetaldehyde, heptane, and acetaldehyde.

10. The composition of claim 1, wherein the composition comprises less than or equal to about 4% by weight of water.

11. The composition of claim 1, wherein the composition comprises less than or equal to about 2% by weight of water.

12. The composition of claim 1, wherein the composition comprises less than or equal to about 1.5% by weight of water.

13. The composition of claim 10 or 12, wherein a weight of water is determined by Karl Fisher analysis.

14. A pharmaceutical composition comprising, carbidopa, entacapone, and greater than 0.15% by weight of Compound 1: 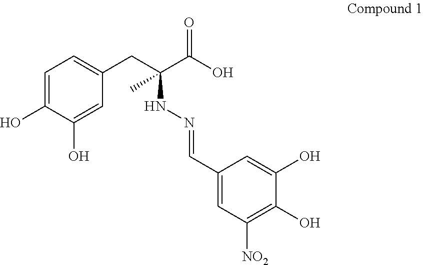 [SMILES: C[C@@](Cc1ccc(O)c(O)c1)(N/N=C/c1cc(O)c(O)c([N+](=O)[O-])c1)C(=O)O]
or a pharmaceutically acceptable salt thereof.

15. The pharmaceutical composition of claim 14, further comprising levodopa.

16. The pharmaceutical composition of claim 14 or 15, characterized in that the pharmaceutical composition is stable for 16 weeks or longer when stored at about 5° C.

17. The pharmaceutical composition of claim 14 or 15, characterized in that the pharmaceutical composition is stable for 16 weeks or longer when stored at 5° C.±3° C.

18. The pharmaceutical composition of claim 14, characterized in that the pharmaceutical composition is stable for 16 hours or longer at physiological temperature (e.g., 37° C.).

19. The pharmaceutical composition of claim 14, wherein the pharmaceutical composition comprises:
about 1-15% by weight of levodopa;
about 0.1-2% by weight of carbidopa; and
about 1-7.5% by weight of entacapone.

20. The pharmaceutical composition of claim 14, wherein the pharmaceutical composition comprises:
about 2% by weight of levodopa;
about 0.5% by weight of carbidopa; and
about 2% by weight of entacapone.

21. The pharmaceutical composition of claim 14, further comprising one or more pharmaceutical carriers.

22. The pharmaceutical composition of claim 21, wherein the one or more pharmaceutical carriers is a polysaccharide.

23. The pharmaceutical composition of claim 22, wherein the polysaccharide is sodium carboxymethyl cellulose.

24. The pharmaceutical composition of claim 22, wherein the pharmaceutical composition comprises about 2-3% by weight of the sodium carboxymethyl cellulose.

25. The pharmaceutical composition of claim 14, wherein the pharmaceutical composition is in the form of a gel.

26. The pharmaceutical composition of claim 25, wherein the pH of the gel is less than about 5.7.

27. The pharmaceutical composition of claim 25 or 26, wherein the pH of the gel is about 4.5 to about 5.5.

28. The pharmaceutical composition of claim 25, wherein the pH of the gel is about 5.0.

29. The pharmaceutical composition of claim 25, wherein the viscosity of the gel is from about 3000 to about 5000 CPS.

30. The pharmaceutical composition of claim 25, wherein the viscosity of the gel is from about 3500 to about 4500 CPS.

31. The pharmaceutical composition of claim 25, wherein the gel is formulated to be administered intestinally.

32. The pharmaceutical composition of claim 14, wherein the pharmaceutical composition is in the form of an oral dosage.

33. A method of treating Parkinson's Disease (PD) in a patient comprising administering to the subject a pharmaceutical composition of claim 14.

34. The method of claim 33, wherein the Parkinson's Disease is Advanced Parkinson's Disease.

35. The method of claim 33 or 34, wherein the pharmaceutical composition is administered to the patient over a 16 hour course.

36. The method of claim 33, wherein about 25% of a total daily dose of the pharmaceutical composition is administered to the patient in the morning.

37. The method of claim 33, wherein the pharmaceutical composition is administered to the patient once daily.

38. The method of claim 33, wherein the pharmaceutical composition is administered directly to the intestine.

39. The method of claim 38, wherein the pharmaceutical composition is administered by a PEG-J tube.

40. A method of treating Parkinson's Disease (PD) in a patient comprising administering to the subject a pharmaceutical composition of claim 32.

41. The method of claim 40, wherein the Parkinson's Disease is Advanced Parkinson's Disease.

42. A method of manufacturing a composition comprising Compound 1 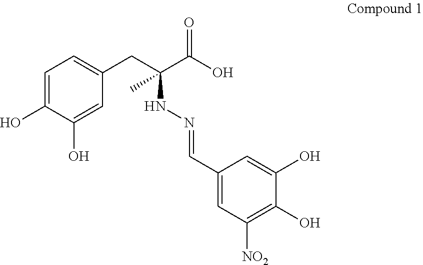 [SMILES: C[C@@](Cc1ccc(O)c(O)c1)(N/N=C/c1cc(O)c(O)c([N+](=O)[O-])c1)C(=O)O]
comprising contacting Compound 2 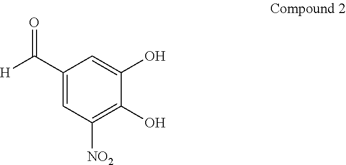 [SMILES: O=Cc1cc(O)c(O)c([N+](=O)[O-])c1]
with Compound 3 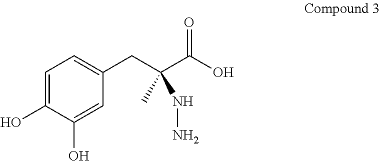 [SMILES: C[C@@](Cc1ccc(O)c(O)c1)(NN)C(=O)O]
in the presence of an organic solvent and a sulfate salt.

43. The method of claim 42, wherein the sulfate salt is sodium sulfate, potassium sulfate, magnesium sulfate, calcium sulfate, or ammonium sulfate.

44. The method of claim 42 or 43, wherein the sulfate salt is sodium sulfate.

45. The method of claim 42, wherein the organic solvent comprises at least one of methanol, ethanol, propanol, isopropanol, and butanol.

46. The method of claim 42, wherein the organic solvent is ethyl acetate.

47. The method of claim 45, wherein the organic solvent is methanol.

48. The method of claim 42, further comprising suspending Compound 1 in an organic solvent, and filtering a solid form of Compound 1, to thereby provide a composition comprising Compound 1 that is greater than 90% by weight free from impurities.

49. The method of claim 48, wherein the composition comprising Compound 1 is greater than 95% by weight free from impurities.

50. The method of claim 49, wherein the composition comprising Compound 1 is greater than 99% by weight free from impurities.

51. The method of claim 50, wherein the composition comprising Compound 1 is substantially free from impurities.

52. The method of claim 42, wherein the organic solvent is dichloromethane.

53. A method of characterizing the purity of a pharmaceutical composition comprising levodopa, carbidopa, and entacapone, the method comprising determining a quantity of Compound 1: 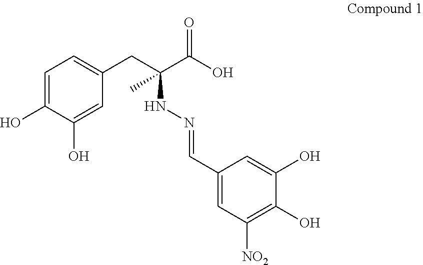 [SMILES: C[C@@](Cc1ccc(O)c(O)c1)(N/N=C/c1cc(O)c(O)c([N+](=O)[O-])c1)C(=O)O]
in the pharmaceutical composition by comparison of a sample of the pharmaceutical composition to a reference standard, wherein the reference standard comprises a known quantity of Compound 1.

54. The method of claim 53, wherein the pharmaceutical composition is characterized as being high purity if the pharmaceutical composition comprises less than about 0.15% by weight of Compound 1.

55. The method of claim 53 or 54, wherein the reference standard is a composition comprising 95% or greater by weight of Compound 1.

56. The method of claim 53, wherein the purity of the pharmaceutical composition is assessed by high pressure liquid chromatography (HPLC) using:
a Waters SunFire C18 Column, 150×4.6 mm, 3.5 um, UV/Vis detector;
a mobile phase A that is 0.06% triflouroacetic acid (TFA) in water; and
a mobile phase B that is 0.04% TFA in (80:20 MeOH;H₂O).

57. A method of using a reference standard comprising a known quantity of Compound 1: 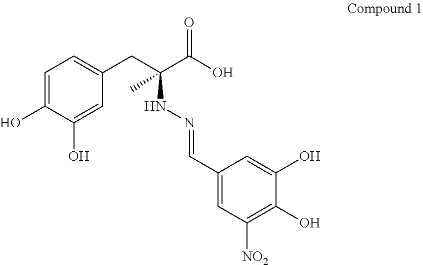 [SMILES: C[C@@](Cc1ccc(O)c(O)c1)(N/N=C/c1cc(O)c(O)c([N+](=O)[O-])c1)C(=O)O]
to determine a level of purity of a pharmaceutical composition, the method comprising comparing a quantity of Compound 1 in the reference standard to a quantity of Compound 1 in the pharmaceutical composition.

58. The method of claim 57, wherein the purity of the pharmaceutical composition is assessed by high pressure liquid chromatography (HPLC) using:
a Waters SunFire C18 Column, 150×4.6 mm, 3.5 um, UV/Vis detector;
a mobile phase A that is 0.06% triflouroacetic acid (TFA) in water; and
a mobile phase B that is 0.04% TFA in (80:20 MeOH;H₂O).
